(19) United States
(12) Patent Application Publication
(10) Pub. No.: US 2007/0100210 A1
(43) Pub. Date: May 3, 2007

(54) Illuminated surgical access system including a surgical access device and coupled light emitter

(75) Inventors: Sean Selover, Westport, MA (US); Sara Dziedzic, Braintree, MA (US); Steve Connolly, Sharon, MA (US)

Correspondence Address: LAHIVE & COCKFIELD, LLP, ONE POST OFFICE SQUARE, BOSTON, MA, 02109-2127, US

(73) Assignee: DePuy Spine, Inc., Raynham, MA (US)

(21) Appl. No.: 11/265,879

(22) Filed: Nov. 2, 2005

(51) Int. Cl. A61B 1/267

(52) U.S. Cl. 600/199

(57) ABSTRACT

A surgical access system for providing access to a surgical site in a patient includes a surgical access device defining a working channel for accessing a surgical site and an integrated annular light emitter for illuminating the working channel. The integrated annular light emitter is coupled to a proximal end of the surgical access device and includes a central opening aligned with the working channel. The integrated annular light emitter includes a light transmission path formed about the central opening that emits circumferential light transmitted from a light source about the circumference of the surgical access device. The light emitter may emit light directly into the interior of the surgical access device, or into a smooth, light-transmitting portion of the side wall of the surgical access device to illuminate a working space.

### FIELD OF THE INVENTION

The present invention relates to devices used in surgery. More particularly, the present invention relates to instrumentation and a method for the providing access and illumination for surgical sites, implements and implants.

### BACKGROUND OF THE INVENTION

In invasive surgical procedures, illumination of a working space may be required to facilitate use of the surgical instruments. For example, in spinal surgery, access ports, comprising generally tubular, open-ended structures, are often used to provide access to a surgical site. The access ports may require illumination at the distal end thereof to facilitate the surgical procedure.

Achieving proper illumination of a surgical site during minimally invasive surgery can be difficult. In the current state of the art, external light sources are used to provide illumination to access ports. However, external light sources are unwieldy, and the link used to transmit the generated light to the access port can be cumbersome and block access by a surgeon to the port. For example, a surgeon may wear a head-mounted light in order to illuminate the working area at the base of an access port. Head-mounted light sources may require the surgeon to constantly direct the light with his or her head at an optimal angle, into the access port in order to view the working area. In addition, fiber optic cables, attached to the light, can encumber the surgeon and tether him or her to a light source.

Another option currently used by surgeons involves lights mounted on an overhead microscope. When using an overhead microscope, the light source is distant from the surgical site, increasing the likelihood of creating shadows and potentially obstructing the ability of the light to reach the working area.

Other alternatives for lighting a surgical site place small light sources into the interior of an access port to illuminate the work space. However, the use of a light source within the access port can reduce the available working area in the port, and may hinder the use of instruments that enter and exit the port during surgery.

### SUMMARY OF THE INVENTION

The present invention provides an illuminated surgical access system including a light emitter coupled to a surgical access device. The surgical access device defines a path or port to a surgical site and the light emitter emits and directs light into and along the path to illuminate a surgical site accessed by the surgical access device. The integrated light emitter is coupled to a proximal end of the access device to provide illumination of the working site and provides circumferential light about an interior path through the surgical access device and/or through the side walls of the surgical access device. The integrated light emitter may comprise an annular ring forming a light transmission path that provides light to the interior of the surgical access device. The integrated light emitter does not reduce the working area of the access device or hinder the surgeon, while providing superior illumination of a surgical site.

According to a first aspect of the invention, an illuminated surgical access system for providing access to a patient during surgery comprises a surgical access device comprising at least one sidewall and defining an interior path therethrough forming a port for accessing the patient; and a light transmission path defined about at least a substantial portion of an inner circumference of a proximal end of the surgical access device, the light transmission path overlapping the interior path of the surgical access device for transmitting light from a light source to the interior of the access device.

According to another aspect of the invention, an illuminated surgical access system for providing access to a surgical site of a patient during surgery comprises a tubular surgical access device comprising at least one smooth side wall and defining an interior path therethrough forming a port for accessing the surgical site and an illuminating light ring coupled to a proximal end of the surgical access device for transmitting light from a light source to the surgical site.

According to another aspect of the invention, a light emitter configured to couple to a proximal end of an access device comprises a ring-shaped housing that receives a light-transferring cable connected to a light source, a central hole formed by the ring-shaped housing, a light transmission path formed about the central hole and coupling means disposed radially outward of the light transmission path for receiving a side wall of a surgical access device.

According to still another aspect of the invention, a method of accessing a surgical site in a patient comprises the steps of providing an access device comprising at least one sidewall having an elongated channel and defining a path therethrough forming a port for accessing the patient, powering a light source to produce light that is transmitted through a light transmission path formed about an inner circumference of the access device and directing the light through the access device towards the surgical site.

### BRIEF DESCRIPTION OF THE FIGURES

These and other features and advantages of the present invention will be more fully understood by reference to the following detailed description in conjunction with the attached drawings in which like reference numerals refer to like elements through the different views. The drawings illustrate principals of the invention and, although not to scale, show relative dimensions.

FIG. 1 illustrates an illuminated surgical access system including a light emitter coupled to a proximal end of an access device according to an embodiment of the invention.

FIGS. 2A-2C illustrate an illuminated surgical access system including a light emitter comprising a light-emitting cap according to one embodiment of the invention.

FIG. 3 is a cutaway perspective view of the illuminated access system of FIGS. 2A-2C.

FIGS. 4A and 4B illustrate the light-emitting cap of the illuminated surgical access system of FIGS. 2A-2C.

FIG. 5 illustrates an embodiment of an access device for an illuminated surgical access system including snap-on tabs for coupling the access device to a light emitter.

FIG. 6 illustrates an embodiment of a light-emitting cap include mirrored surfaces for directing light through the light emitting cap.

FIGS. 7A-7B another embodiment of light emitter comprising a ring of fiber optic cables embedded in a housing.

FIG. 8 illustrates an embodiment of a surgical access system including a ring of fiber optic cables extending through a tubular access device housing.

FIGS. 9A-9C illustrates an embodiment of a surgical access system including a molded light emitter molded onto a proximal end of a surgical access device.

FIGS. 10A-10B illustrate another embodiment of a surgical access system including a molded light emitter molded onto a proximal end of a surgical access device.

### DETAILED DESCRIPTION OF THE INVENTION

The present invention provides an improved surgical access system for accessing a surgical site during surgery including an integrated light emitter for illuminating the surgical site. The present invention will be described below relative to certain illustrative embodiments. Those skilled in the art will appreciate that the present invention may be implemented in a number of different applications and embodiments and is not specifically limited in its application to the particular embodiments depicted herein.

The illuminated surgical access system of the illustrative embodiment of the invention may be used in spinal surgery, for example, during a discectomy or microdiscectomy procedure to remove damaged disc material from the spine, though one skilled in the art will recognize that the invention can be used with any surgical instrument in any surgical procedure that requires illumination. Examples of surgical procedures suitable for employing the illuminated surgical access system of the present invention include, but are not limited to, insertion of interbody fusion devices, bone anchors, fixation devices, including rods, plates and cables, artificial disks, hip stems, artificial ligaments, trochars for gastro-intestinal work, or any procedure requiring access to a patient as well as visualization. The surgical access system may be part of any suitable implant instrument used to provide access to a particular area of a patient's body where visualization is also needed. The surgical access system can be used to position any suitable implant, instrument and/or other device in any suitable procedure where guidance of the implant, instrument and/or device is used. Alternatively, or in addition to providing guidance, the surgical access system may be used to dilate a surgical incision using a set of progressively larger cannulas or an expanding cannula to provide access to a surgical site.

An illustrative embodiment of the present invention provides a lighted, minimally invasive access to a surgical site via a low profile port that used integrated light transmission and emission technology. The invention facilitates access to a surgical site without decreasing a working space in the port or requiring extra equipment to be worn by the surgeon.

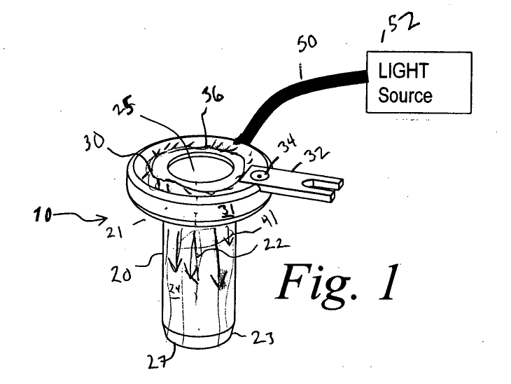

FIG. 1 illustrates an illuminated surgical access system of an illustrative embodiment of the invention for providing both access and illumination of a surgical site during performance of a surgical procedure. The illustrative illuminated surgical access system 10 includes a port, illustrated as an access device 20 comprising a substantially hollow tubular body, for accessing a surgical site, and a light emitter 30 coupled to a proximal end 21 of the surgical access device 20 for illuminating an interior path 22 extending through the access device 20. A light transferring cable 50 or other suitable light transmission means transmits light from a light source 52 to the light emitter 30, which emits and directs the light 41 along the path 22. The light emitter 30 may also or alternatively emit light into and through a transparent portion of the access device.

The illustrative access device 20 may be a standard access port, in the shape of a cannula comprising a hollow tubular body suitable for insertion in and/or placement adjacent to a patient's body. The illustrative access device 20 has at least one hollow channel or lumen defining an interior path 22 extending from an open proximal end 21 of the access device to an open distal end 23 of the access device. The path 22 may form a working channel or at least a portion of a working channel for accessing a surgical site adjacent to or in the vicinity of the distal end 23 of the tubular body. In the illustrative embodiment, the body of the access device 20 includes open proximal end 21 that forms a proximal port 25 of the interior path 22, and the open distal end 23 forms a distal port 27 of the interior path 22 for allowing access to the surgical site. One skilled in the art will recognize that the access device 20 may have any suitable configuration and size for providing access to an area of a body. The illustrative access device may be used for retaining soft tissue away from a surgical site and/or guiding a surgical instrument, device and/or implant, though one skilled in the art will recognize that the access device may comprise any suitable device defining a path or channel requiring illumination.

As shown, the tubular body of the illustrative access device 10 is formed by a cylindrical sidewall 24 preferably having smooth inner and outer surfaces, though one skilled in the art will recognize that the tubular body can have any size, shape, configuration and number of sidewalls. The side wall 24 may be opaque or transparent to light. The access device can be any suitable device defining a port for providing access to a surgical site. The access device can have any suitable cross-section and is not limited to the cylindrical cross-section shown in the illustrative embodiments. The access device can be open or closed to define an open or closed path therethrough.

The surgical access device 20 can be formed of any suitable surgical material, such as, but not limited to, plastic, surgical stainless steel and other materials known in the art. An example of a suitable material is acrylic polycarbonate, though other materials may also be used.

The light emitter 30 coupled to the proximal end 21 of the access device 20 illuminates the path 22 through the distal port 27. Preferably, the light emitter 30 emits circumferential light into the interior of the access device 20 directly inside and about the inner circumference of the proximal end 21, or about at least a substantial portion of the inner circumference. For example, for an access device that has an open side, the light emitter 30 may distribute light about perimeter of the side wall and may or may not distribute light where the side wall of the access device is open. Similarly, for an arc-shaped access device, the inner circumference refers to the inner edge of the arc and is not required to be a full circle or loop. The light 41 emitted into the tube interior is directed through path 22, out distal port 27 and into a working space adjacent to the distal port 27.

Preferably, the light emitter 30 comprises a housing 31, which may be in the shape of an annular ring, as shown, to leave the proximal port 25 of the access device 20 open, and at least one light transmission element 36 disposed in the housing for emitting light transferred to the light transmission element 36 from a light source 52 via the intermediate light transmission means 50. Preferably, the light transmission element 36 emits light circumferentially about the inner perimeter of the side wall 24.

The light source 52 may be any suitable device for producing light, including, but not limited to, halogen light boxes, incandescent light boxes and other light sources readily available in most hospital settings, such as those available from Welch Allyn Medical Products of Skaneateles Falls, N.Y. The light source may have any suitable power level. In an illustrative embodiment, the light source is a 300 Watt Halogen Light Box. Any other suitable light source capable of producing light that is transmitted to the light emitter 30 via the light transferring cable 50, which may be fiber optic cables or any other suitable light transmitter, may also be used.

The light emitter 30 can be integrally formed with or removably coupled to the proximal end 21 of the access device 10. The light emitter may be coupled to the proximal end through any suitable means known in the art, including, but not limited to, press fit, a snap fit, a threaded connection, a luer connection, a bayonet connection or any other suitable means known in the art.

As shown in FIG. 1 the light emitter 30 may include a rigid arm attachment 32 for attaching the light emitter 30 and an access device 20 coupled thereto to a base or other suitable system for allowing positioning of the illuminated surgical access system relative to a selected surgical site. As show, a fastening device 34 may be provided for coupling the body of the light emitter 30 to the arm 32, though one skilled in the art will recognize that any suitable means may be used for coupling the tubular body to a base or other system may be used.

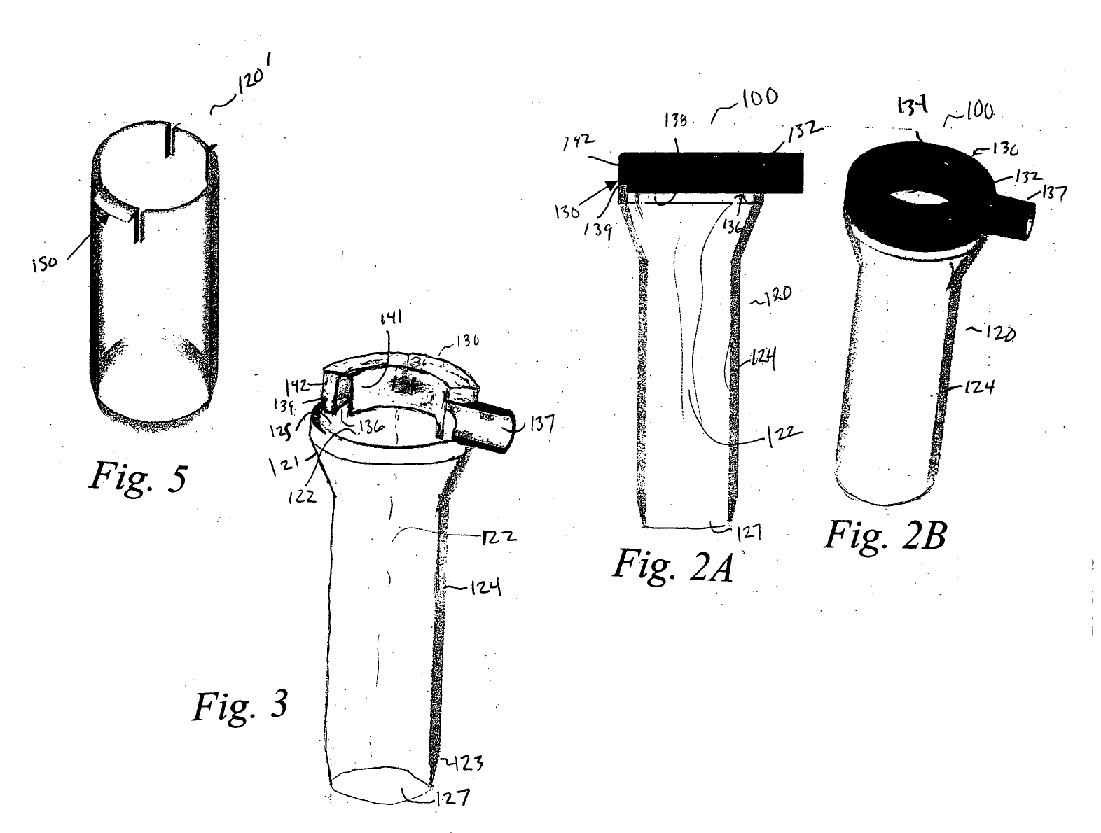

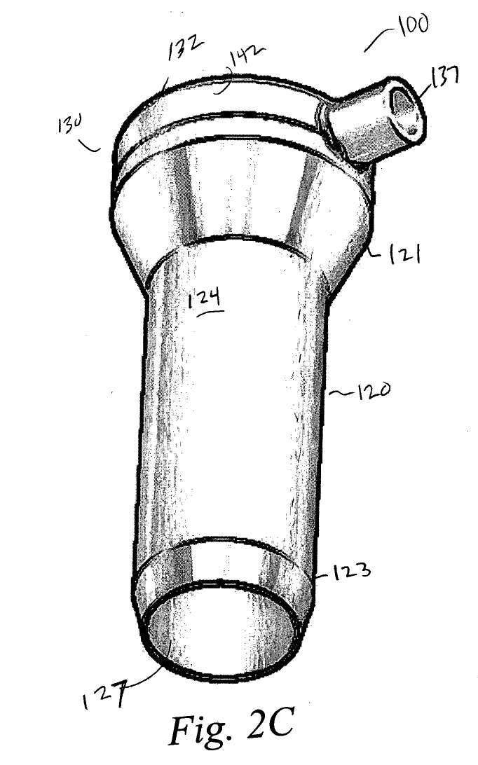

The light emitter 30 coupled to the proximal end of the surgical access device may have any suitable size, shape and configuration. For example, FIGS. 2A-2C illustrate an illuminated surgical access system 100 including a light emitter 130 coupled to a proximal end of a surgical access device 120 according to one embodiment of the invention. The illustrative light emitter 130 comprises a circumferential cap, illustrated as a ring-shaped light-emitting cap 132, configured to snap onto, thread onto, or otherwise engage the proximal end of a tubular surgical access device 120. Preferably, the light-emitting cap removably engages the tubular body 124 of the surgical access device 120.

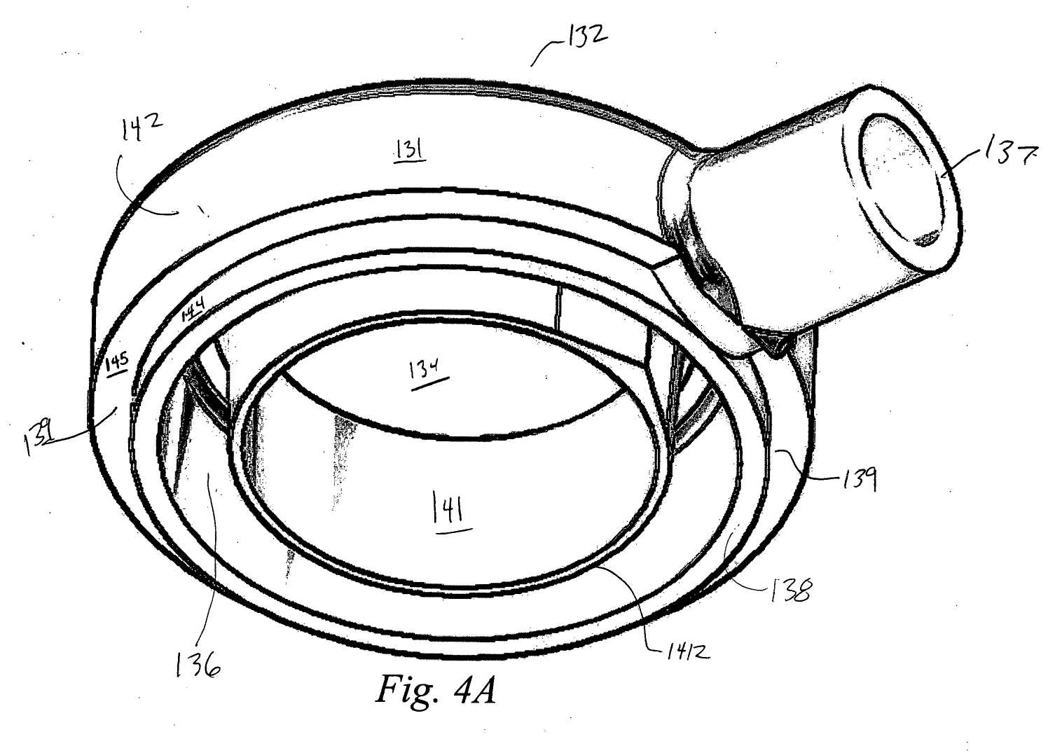

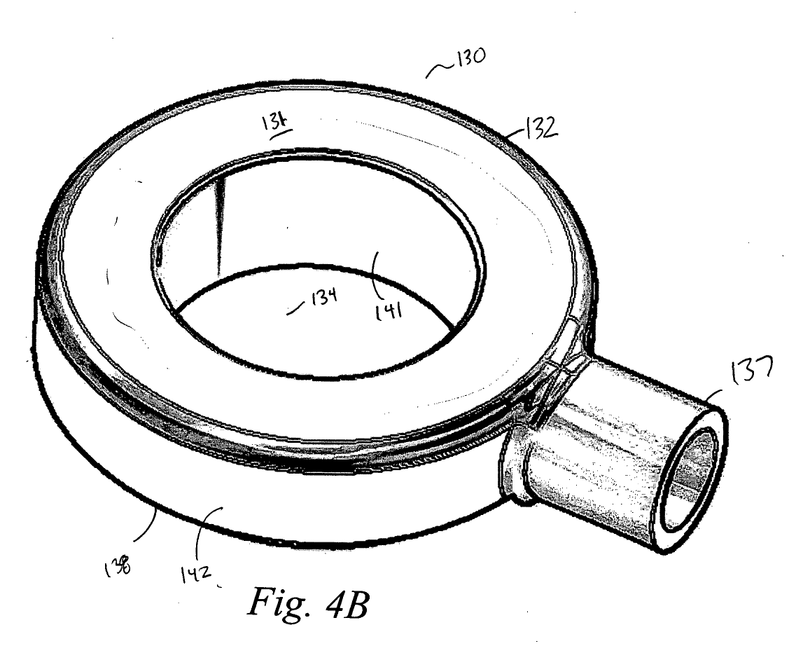

The light-emitting cap 132, shown in detail in FIGS. 3, 4A and 4B, comprises an annular housing 131 defining a central opening 134, which forms a proximal end of the working channel 122 when assembled. When the light-emitting cap 132 is coupled to the proximal end of the access device 120, the central opening 134 preferably aligns with the distal port 127 of the access device to define both ends of the working channel 122, as described in detail below.

The ring-shaped, light emitting cap 132 is not limited to an annular or circular shape and may have any suitable shape that matches the circumference of a corresponding surgical access device.

Referring to FIGS. 3-4B, the interior of the ring-shaped light-emitting cap 132 includes a circumferential light transmission path 136 formed about the central opening 134 forming a light transmission element for transmitting and emitting light to illuminate the surgical access device 120. The illustrative light transmission path 136 comprises an annular groove or slot formed on the bottom surface 138 of the light-emitting cap 132 capable of transmitting light therethrough. The annular groove or slot is filled with a light-transmitting medium, such as air or another transparent material, so that light is distributed about the light transmission path. The open or otherwise transparent bottom of the light transmission path allows light to be emitted from the light transmission path in a downward direction. As shown, an annular wall 141 defining the central opening 134 also forms an inner side wall of the light transmission path 136.

As described below, the central opening 134 and light transmission path 136 preferably have a smaller diameter than the diameter of the proximal end 121 of the corresponding surgical access device, such that the open bottom of the light transmission path 136 overlaps the interior 122 of a corresponding surgical access device when ring-shaped light-emitting cap 132 is coupled to the tubular surgical access device 120. Light provided to the light transmission path 136 is directed through the light transmission path and down into the interior of the surgical access device.

In the illustrative embodiment, the outer side wall 142 of the light transmission path receives the side wall 124 of the corresponding access device, such that the light transmission path 136 aligns with the inside surface of the side wall 124, causing that light transmitted through the path to be directed into the interior 122 of the access device.

Preferably, the light transmission path 136 includes one or more chamfered edges to facilitate channeling of light downward into the interior of the access device 20. The illustrative chamfered edges 1412 are formed at the end of the annular wall 141 to enhance light direction down through the working channel 22. The chamfered edges may extend at any suitable angle. In the illustrative embodiment, the chamfered edge is between about 30 and about 60 degrees relative to the longitudinal axis of the surgical access device, and preferably about 45 degrees relative to the longitudinal axis, though one skilled in the art will recognize that the edge of the annular wall 141 may be chamfered at any suitable angle.

A peripheral opening 137 provides access to the light transmission path 136 from an exterior surface of the light-emitting cap. The peripheral opening 137 allows docking or connection of a fiber optic or other light transferring cable, which may connect the cap 132 to a light source. Light transferred from the light source through the light transferring cable passes into the light transmission path 136 via the peripheral opening 137. The light transmission path 136 disperses the light about the inner circumference of the access device and into the path 122.

The light-emitting cap 132 further includes a coupling mechanism for coupling the proximal end 121 of the tubular body 120 to the light emitter 130. As shown, the illustrative cap includes a groove comprising a circumferential step 139 sized and configured to receive the proximal end 121 of the side wall 124 of the tube forming the access device 120. The side wall-receiving step 139 is disposed radially outward of the light transmission path 136, so that the light transmission path is formed over the interior of the tube when the illuminated surgical access system 100 is assembled. In the illustrative embodiment, the side wall-receiving step 139 comprises horizontal wall 145 and vertical wall 144 and is formed at the intersection between the outer side surface 142 and the bottom surface 138 of the cap 132, so that the outer surface of the access device side wall 124 aligns with the outer side surface 142 of the light emitting cap 132.

The resulting structure may be boss-shaped, with the wall 145 receiving a proximal top surface of the access device, wall 144 extending into interior of the access device and abutting inner surface of the side wall 124, and light transmission path 136 formed about the inner circumference of the tubular body, providing even, circumferential light about the interior of the access device.

In the embodiment of FIGS. 2A-3, the proximal port 125 of the tubular surgical access device 120 is wider than the distal port 127, and the side wall tapers 124 from the proximal end 121 towards the distal end 123. In this manner, the integration of the light-emitting cap 132 at the proximal end does not reduce the working space of the access device. The inner diameter 124 of the ring preferably aligns with the inner diameter of the distal end 123 of the tubular access device.

According to another embodiment, the coupling mechanism can comprise an annular slot or recess formed in the bottom surface 138 disposed between the side wall 142 and the light transmission path 136, such that the outer side surface 145 of the light-emitting cap 132 is wider than the outer surface of the side wall 124.

Alternatively, at least a portion of the light transmission path 136 may align with the proximal edge of the side wall 124 of a corresponding surgical access device (i.e., the diameter of the light transmission path is about the same as the diameter of the proximal end of the surgical access device). In this embodiment, the side wall of the surgical access device preferably is formed of or includes a light-transmitting material, so that light from the light transmission path diffuses through the access device material and is emitted from the distal end of the side wall to illuminate the working space created by the access device. Alternatively, the light is focused into the interior path 122 of the surgical access device from the side wall 124. When light is transmitted through the side wall, the side wall 124 preferably has a smooth inner surface with no deformities.

In another embodiment of the invention, shown in FIG. 5, a surgical access device 120′ may include tabs 150 or another suitable mechanism to facilitate engagement with a light emitter, such as the ring-shaped light emitting cap 132 of FIGS. 4A and 4B. The flexible tabs 150 are formed at the proximal end of the access device 120′ and are configured to engage corresponding recesses in the light emitter. The flexible tabs allow for the light-emitting cap to snap onto the access device and retain the access device in a corresponding slot.

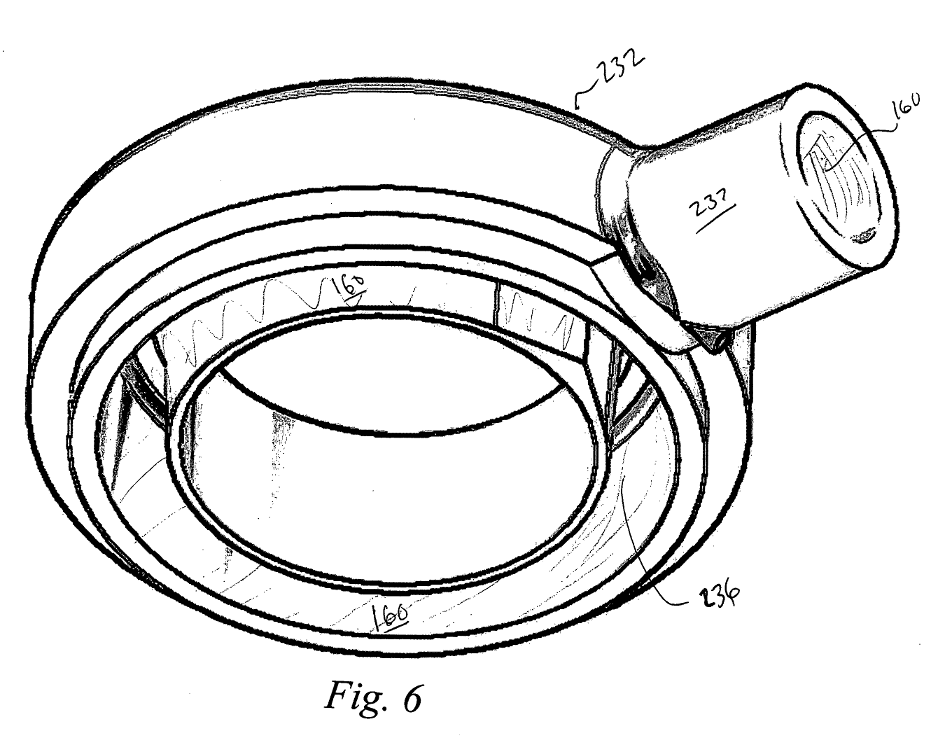

According to one embodiment of the invention, shown in FIG. 6, reflective or mirrored surfaces 160 in the light transmission path 236 on the underside of the light-emitting cap 232 and/or at opening 237 may be provided. The mirrored surfaces 160 channel light from a light transferring cable docked in the opening 237 to the inner diameter of the light-emitting cap 232, and then downward into the working space of a corresponding surgical access device. The mirrored surfaces 160 can be polished metal inserts, formed of chrome or another suitable metal, reflective coatings or any other suitably reflective material.

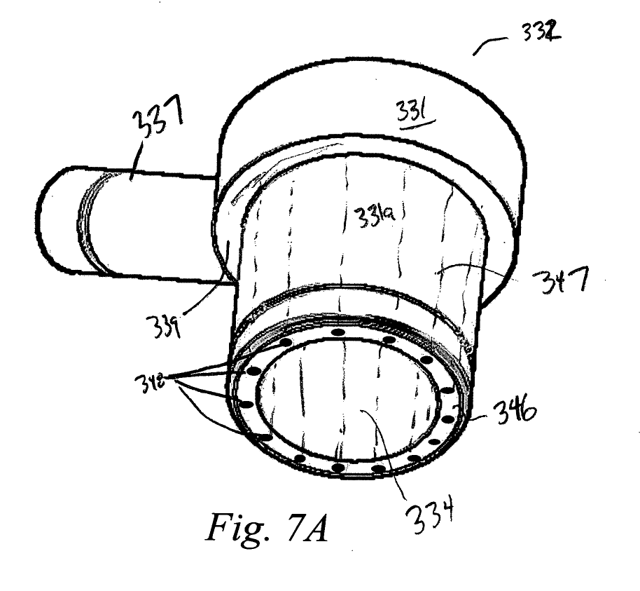

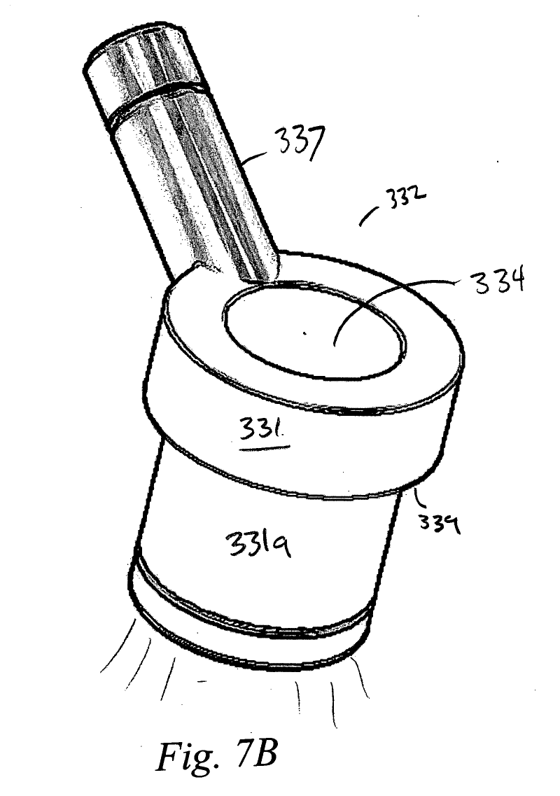

FIGS. 7A-7B illustrate another embodiment of a light-emitting ring-shaped cap 332 forming a light emitter that may be integrated with a surgical access device according to another embodiment of the invention. In the embodiment of FIGS. 7A-7B, a light transmission path 346 for transmitting and emitting light circumferentially about the interior of a surgical access device comprises a number of point sources, illustrated as embedded fiber optic strands 347 distributed about the periphery of the central opening 334 of the light-emitting ring-shaped cap 332. As shown, each fiber optic strand 347 terminates within the light transmission path 346 in a downward direction. The fiber optic strands 347 may be bundled together within a light transferring cable or other suitable light transmission means connected to a light source. The light transferring cable passes through opening 337 in the light-emitting cap 332 and the fiber optic strands 347 branch off about the periphery of the central opening 334 to form the light transmission path 346.

When the fiber optic strands 347 are connected to a light source, the termination points 348 of the fiber optic strands 347 emit light to illuminate the inner diameter of the surgical access device, providing an even circumferential light source inside the access device. The termination points 348 may terminate at an angle to facilitate direction of light emitted from the fiber optic strands.

Alternatively, each fiber optic strand 347 may include a modified cladding or other suitable means at selected locations to provide light emission points within the light transmission path 346 at an intermediate location along the length of the fiber optic strand, rather than solely at a termination point.

In one embodiment, the fiber optic strands 347 may be encased in the body 331 of the cap 332. The cap may be formed of or include a light-transmitting material over the termination points 348 or other emission points to transmit light emitted from the termination points 248 or other emission points to the access device interior. Alternatively, the termination points 348 or other emission points may be exposed.

The illustrative light-emitting cap 332 is substantially boss-shaped, with the light transmitting path forming an annular protrusion 33 la extending from the main body 331 of the cap. The annular protrusion 331 a has a smaller outer diameter than the main body 331 to form a step 339 radially outwardly of the light transmission path 36 for receiving a side wall of a corresponding access device. When assembled, the annular protrusion 33 la of the housing 331 extends into the interior of the corresponding surgical access device to distribute light peripherally about the interior of the surgical access device.

The illustrative light-emitting cap 332 may employ the use of a snap lock attachment, recess or other suitable means for connection to a corresponding surgical access device.

Alternatively, the emission points of the light-emitting fiber optic strands may align with the proximal edge of the side wall of a corresponding surgical access device (i.e., the diameter of the light transmission path is about the same as the diameter of the proximal end of the surgical access device). In this embodiment, the side wall of the surgical access device preferably is formed of or includes a light-transmitting material, so that light from the light transmission path diffuses through the access device material to the distal end of the side wall and eventually illuminates the working space created by the access device, or the light is focused through the side walls to illuminate a workspace created by the surgical access device. When light is transmitted through the side wall, the side walls preferably have a smooth inner surface with no deformities.

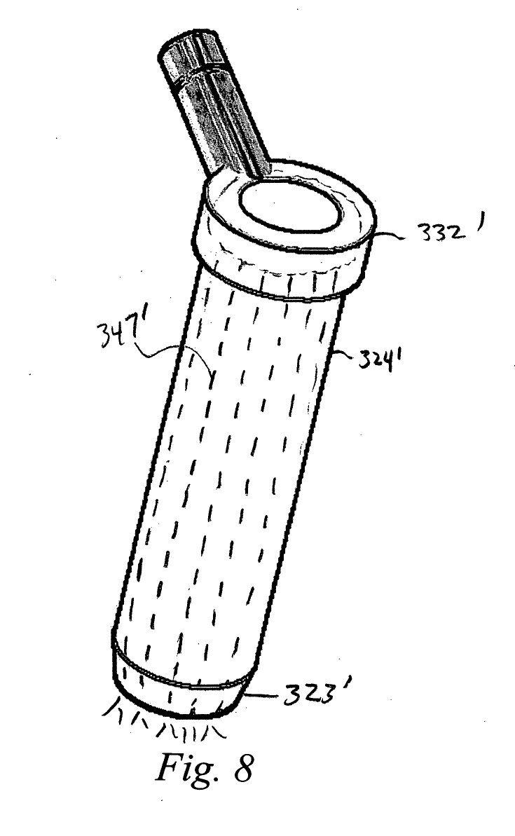

In another embodiment of the invention, shown in FIG. 8, the fiber optic strands 347′ distributed about a light-emitting annular cap 332′ extend through the side wall 324′ of the surgical access device, and emit light out of the distal end 323′.

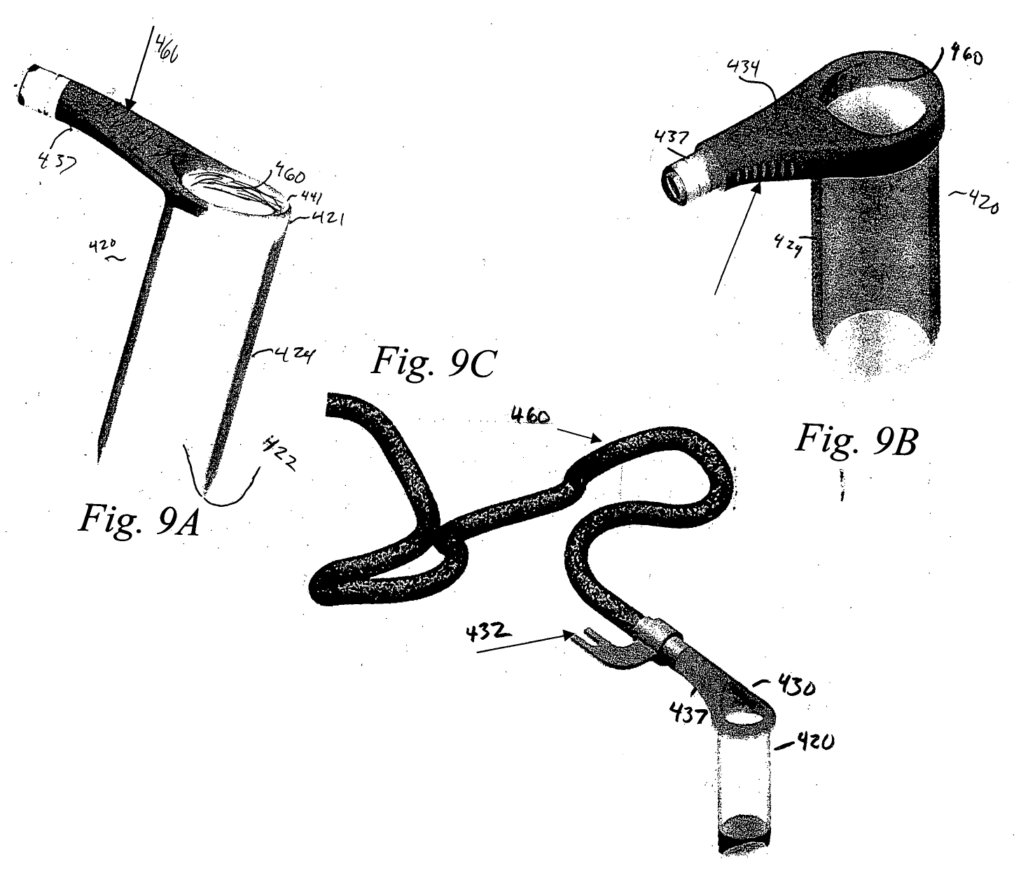

FIGS. 9A-9C illustrate an illuminated surgical access system including a light emitter molded to a proximal end of a surgical access device according to another embodiment of the invention. The surgical access system of FIGS. 9A-9C includes a tubular surgical access device 420 that is molded in a manner such that a substantially cylindrical rod portion 437, shaped as to connect to a light transferring cable, is blended into one side of the proximal end 421 of the access device 420. The molded material of the cylindrical rod portion 437 is a light transferring material or includes a light-transmitting material forming at least a portion of a light transmission path comprising air, plastic or another suitable light-transmitting material. Light entering the cylindrical rod portion 437 is transferred into the hollow tubular portion 422, thus illuminating the working space.

As shown in FIG. 9C, a fiber optic cable 460 may be docked in the cylindrical rod portion 437 to transfer light to the cylindrical rod portion, which distributes the light to the interior of the surgical access device 420.

To enhance light transmission from the cylindrical portion 437 of the tubular access device 420, reflective coatings 460 may be employed on the outer diameter of the cylindrical portion, as well as the first few millimeters of the inner surface of the side wall 424 at the proximal end 421 of the hollow tubular access device.

A chamfered edge 441 along the proximal outer edge of the tubular access device prior may also enhance light direction down into the interior path 422 through the tubular access device.

To prevent reflection of the light into the surgeon's eyes, a second, over-molded piece 434 of non-reflective, opaque material, such as plastic, caps off the tubular access device 420 and cylindrical rod portion 437. The overmolded piece 434 may also include a rigid arm attachment 432 for attaching the light emitter 430 and an access device 420 coupled thereto to a base or other suitable system for allowing positioning of the illuminated surgical access system relative to a selected surgical site, as shown in FIG. 9C.

Alternatively, or in addition to transmitting the interior path through the surgical access device, the light emitter may pass light through the side wall 424 of the surgical access device, which is formed at least partially of a light-transferring material. When passing light through the side wall, the interior surface of the side wall is preferably smooth and free of deformities.

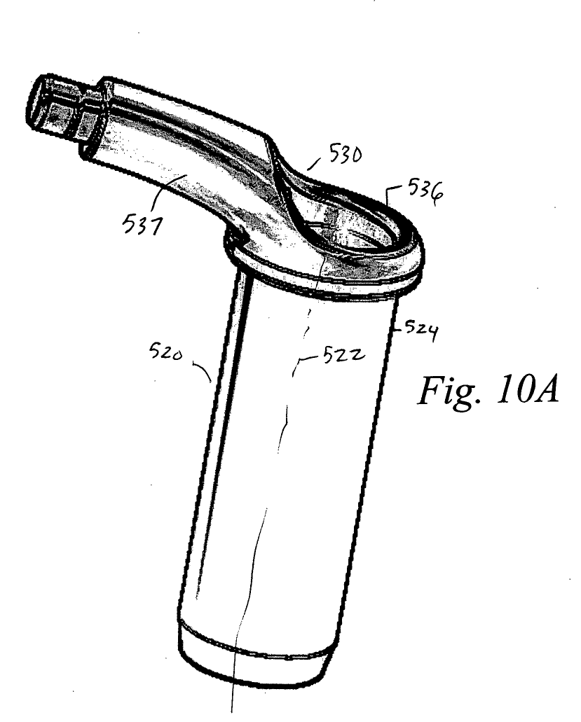

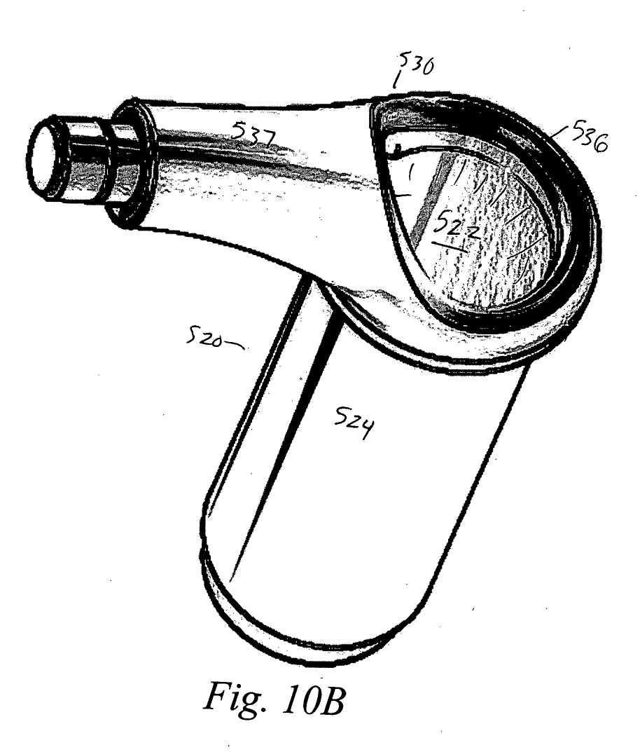

FIGS. 10A and 10B illustrate another embodiment of an illuminated surgical access system including a proximal light emitter. The embodiment of FIGS. 10A and 10B comprises a tubular surgical access device 520 including a molded light emitter 530 molded to the proximal end 521. The molded light emitter 530 includes a peripheral port 537 for receiving a light-transmitting cable and a circumferential light transmission path 536 for distributed light provided by a light-emitting cable to the inner circumference of the surgical access device. Alternatively, or in addition, the circumferential light transmission path 536 may transfer light into the smooth side wall 524, which transfers and emits the light from the distal end or into the interior 522 of the surgical access device at an intermediate location.

The molded light emitter 530 may include an opaque over-molded piece, or may comprise a single molded piece forming and enclosing the proximal end of the light transmission path 536.

As described above, the access device 10 forming the illuminated surgical access system provides working channel from the skin proximate vertebrae to perform a procedure at or near the vertebrae. An example of a suitable access device is the PIPELINE® discectomy port available from DePuy Spine, Inc of Raynham, Mass., though any suitable access device may be used.

The access device 20 can be spaced from or directly interface with a surgical site. The distal end 27 of the tubular body can be configured to interface with bone or another feature to facilitate positioning of the tubular body along a suitable trajectory relative to the surgical site. For example, the distal end 27 may shaped to engage a part of the surgical site, such as a vertebral structure, and can optionally include teeth or other suitable feature formed on an outer surface for engaging a part of the surgical site, such as a vertebra. While the illustrative embodiments show access devices with a flat distal end, the distal end may alternatively be angled or have another suitable shape.

The tubular body of the illustrative access device can be rigid, semi-rigid or flexible, and can have any suitable size, shape and configuration suitable for defining a working channel and/or access to a surgical site. In the illustrative embodiment, the tubular body is straight to define a straight channel therethrough, though one skilled in the art will recognize that the tubular body may define a shaped trajectory therethrough. The tubular body is not limited to a tubular structure having closed sidewalls and can be any component that defines a path, including an open channel or a solid member.

The path through the access device may also or alternatively form a working channel configured to receive selected surgical instruments, such as awls, bone taps, obturators, drills, guide wires, and/or implants, such as screws, fusion devices, artificial disks and hip stems, along the longitudinal axis thereof.

In one embodiment, the illuminated access device 10 may be configured to guide instruments along the working channel. In such an embodiment, the inner diameter of the tubular body may be slightly larger than the outer diameter of the instrument guided by the tubular body, so that the instrument can be inserted through the tubular body while the sidewalls of the tubular body maintain the instrument at a predetermined angle relative to the patient. Alternatively, an instrument to be guided by the tubular body can be configured to slide over the tubular body, with the tubular body maintaining the orientation of the instrument as the instrument slides relative to the tubular body. In this embodiment, the tubular body can have an outer diameter that is slightly less than an inner diameter of an instrument. However, the access device 10 need not form a trajectory or guide for instruments and can be any device suitable for providing access to a surgical site.

The integrated light emitter 30 in the surgical access system of the illustrative embodiment of the invention can comprise any suitable means for producing light that may directly coupled to or integrated with the tubular body of an access device. The integrated light emitter easily integrates illumination into a surgical access device, without requiring cumbersome cables, while allowing direction of light to an ideal location. The integrated light emitter does not compromise or reduce the working area, as it is coupled to a proximal end of the surgical access device and keeps the interior of the surgical access device clear.

The integrated light source provides superior illumination by providing even, circumferential light distributed about the periphery of a surgical access device, preferably in the interior of the surgical access device. In addition, the light emitter may be easily removed or decoupled from a surgical access device to allow reuse, disposal or cleaning. In this manner, the light emitter of the illustrative embodiments of the invention provides enhanced illumination during many types of surgical procedures.

The light emitter may have any suitable configuration to allow the light emitter to directly couple to the tubular body to integrate the light emitter into the access device.

One skilled in the art will recognize that the access device may have any suitable size, shape and configuration for providing access to a surgical site.

The present invention has been described relative to an illustrative embodiment. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

1. An illuminated surgical access system for providing access to a patient during surgery, comprising:
a surgical access device comprising-at least one sidewall and defining an interior path therethrough forming a port for accessing the patient; and
a light transmission path defined about at least a substantial portion of an inner circumference of a proximal end of the surgical access device, the light transmission path overlapping the interior path of the surgical access device for transmitting light from a light source to the interior of the access device.

2. The surgical access system of claim 1, wherein the light transmission path is formed in an illuminating ring-shaped cap coupled to the proximal end of the access device.

3. The surgical access system of claim 2, wherein the illuminating ring-shaped cap includes a rigid arm attachment for attaching the surgical access system to system for allowing positioning of the surgical access system relative to a selected surgical site.

4. The surgical access system of claim 2, wherein the illuminating ring-shaped cap further comprises a circumferential step spaced outwardly from the light transmission path for receiving the distal end of the side wall of the access device.

5. The surgical access system of claim 1, wherein the illuminating light ring is molded to the distal end of the access device.

6. The surgical access system of claim 1, further comprising at least one reflective surface formed in the light transmission path for directing light to the interior path of the surgical access device.

7. The surgical access system of claim 1, wherein the light transmission path includes a chamfered edge.

8. The surgical access system of claim 1, wherein the light transmission path comprises a plurality of fiber optic cables.

9. The surgical access system of claim 8, wherein the fiber optic cables terminate about the periphery of the access device for emitting light about the inner circumference of the access device.

10. The surgical access system of claim 1, wherein the proximal end of the surgical access device is wider than a distal end of the surgical access device, such that a central opening of the light emitting cap aligns with the distal end.

11. The surgical access system of claim 1, further comprising an opening in the light transmission path for receiving a light transferring cable coupled to a light source.

12. The surgical access system of claim 1, wherein the light transmission path is molded to the access device.

13. The surgical access system of claim 12, further comprising an overmolded piece covering the light transmission path.

14. An illuminated surgical access system for providing access to a surgical site of a patient during surgery, comprising:
a tubular surgical access device comprising at least one smooth side wall and defining an interior path therethrough forming a port for accessing the surgical site; and
an illuminating light ring coupled to a proximal end of the surgical access device for transmitting light from a light source to the surgical site.

15. The illuminated surgical access system of claim 14, further comprising a circumferential light transmission path defined about a central hole in the illuminating light ring for transmitting light from a light source about the circumference of the illuminating light ring.

16. The illuminated surgical access system of claim 15, wherein the circumferential light transmission path comprises an annular groove formed in a bottom surface of the illuminating light ring.

17. The illuminated surgical access system of claim 16, wherein the circumferential light transmission path overlaps the interior path of the surgical access device for transmitting light from a light source to the interior of the access device.

18. The illuminated surgical access system of claim 15, wherein the circumferential light transmission path includes at least one reflective surface for directing light to the interior path of the surgical access device.

19. The illuminated surgical access system of claim 15, wherein the light transmission path includes a plurality of fiber optic cables having termination points distributed about the periphery of the light transmission path for emitting light into the interior path.

20. The illuminated surgical access system of claim 15, wherein the circumferential light transmission path transmits light from a light source about an circumference of tubular access device and into the smooth side wall of the transmission device.

21. The illuminated surgical access system of claim 15, wherein an inner diameter of the circumferential light transmission path is equal to or less than an outer diameter of the surgical access device.

22. A light emitter configured to couple to a proximal end of an access device, comprising:
a ring-shaped housing that receives a light-transferring cable connected to a light source;
a central hole formed by the ring-shaped housing;
a light transmission path formed about the central hole; and
coupling means disposed radially outward of the light transmission path for receiving a side wall of a surgical access device.

23. The light emitter of claim 22, wherein the coupling means comprises a step formed at an intersection between an outer side surface and a bottom surface of the housing.

24. The light emitter of claim 22, wherein the light transmission path comprises an annular groove formed in a bottom surface of the ring-shaped housing.

25. The light emitter of claim 22, wherein the light transmission path comprises a plurality of fiber optic cables having termination points distributed about the periphery of the central hole.

26. A method of accessing a surgical site in a patient comprising the steps of:
providing an access device comprising at least one sidewall having an elongated channel and defining a path therethrough forming a port for accessing the patient;
powering a light source to produce light that is transmitted through a light transmission path formed about an inner circumference of the access device; and
directing the light through the access device towards the surgical site.
